(19) United States
(12) Patent Application Publication
(10) Pub. No.: US 2018/0289363 A1
(43) Pub. Date: Oct. 11, 2018

(54) SURGICAL ACCESS SYSTEM, DEVICES THEREOF, AND METHODS OF USING THE SAME

(71) Applicants: BARNES, PC, Bryan, Athens, GA (US); K2M, Inc., Leesburg, VA (US)

(72) Inventors: Bryan B. Barnes, Athens, GA (US); Larry McClintock, Gore, VA (US)

(73) Assignee: K2M, Inc., Leesburg, VA (US)

(21) Appl. No.: 15/766,079

(22) PCT Filed: Oct. 6, 2016
(86) PCT No.: PCT/US16/55676

§ 371 Date: Apr. 5, 2018

Related U.S. Application Data
(60) Provisional application No. 62/237,607, filed on Oct. 6, 2015.

(51) Int. Cl. A61B 17/02; A61B 17/70; A61B 17/88

(52) U.S. Cl. CPC A61B 17/025; A61B 2017/0256; A61B 17/88; A61B 17/7076

(57) ABSTRACT

A dilator includes a main body and a cross brace assembly. The main body includes a pair of body members movable in parallel relationship with respect to each other between a closed position and an open position. The cross brace assembly includes two linkage assemblies, with each linkage assembly having a pair of arms arranged in an X configuration. Each arm includes at least one slidable connection point with the respective body member to which the arm is coupled to facilitate movement of the body members relative to each other.

### CROSS-REFERENCE TO RELATED APPLICATIONS

This application claims the benefit of, and priority to, U.S. Provisional Patent Application Ser. No. 62/237,607, filed on Oct. 6, 2015, the entire contents of which are hereby incorporated herein by reference.

### TECHNICAL FIELD

The present disclosure relates generally to surgical access systems, and more particularly, to a surgical access system for accessing, dilating, and retracting tissue in a surgical site, and methods of using the same.

### BACKGROUND

Disease, the effects of aging, or physical trauma resulting in damage to the spine has been treated in many instances by fixation or stabilization of the effected vertebra. A wide variety of spinal fixation apparatuses have been employed in surgical procedures for correcting spinal injuries and the effects of spinal diseases.

After a partial or complete discectomy, the normally occupied space between adjacent vertebral bodies is subject to collapse and/or misalignment due to the absence of all or a part of the intervertebral disc. In such situations, the physician may insert one or more prosthetic spacers between the affected vertebrae to maintain normal disc spacing and/or the normal amount of lordosis in the affected region.

Typically, a prosthetic implant is inserted between the adjacent vertebrae and may include pathways that permit bone growth between the adjacent vertebrae until they are fused together. As is typical, the intervertebral spaces are accessed anteriorly, posteriorly, or laterally. It would be desirable to access the intervertebral spaces via an approach that provides greater access to the surgical area while applying the least amount of stress to the surrounding tissue.

### SUMMARY

In accordance with an aspect of the present disclosure, a dilator includes a main body and a cross brace assembly. The main body includes a pair of body members movable in parallel relationship with respect to each other between a closed position and an open position. The cross brace assembly includes two linkage assemblies, with each linkage assembly having a pair of arms arranged in an X configuration. Each arm includes at least one slidable connection point with the respective body member to which the arm is coupled to facilitate movement of the body members relative to each other.

Each body member may include a post releasably engageable with a retractor device that is configured to move the body members to the open position. In embodiments, each body member includes a flange extending laterally from a proximal portion thereof, and the post extends proximally from the flange. Each post may include a shaft and an enlarged head. Each body member may include a distal portion having a tapered edge.

The dilator may include a threaded pin having an elongated body removably disposed within a throughhole defined in the main body. In embodiments, the elongated body of the threaded pin includes an enlarged proximal end and a threaded distal end that extends distally beyond distal portions of the body members when the threaded pin is disposed in the throughhole. In some embodiments, each body member includes an inner surface having a recess defined therein that together define the throughhole of the main body when in the closed position, and at least one of the recesses is configured to retain the threaded pin therein when the body members are in the open position.

Each body member of the main body may include proximal slots and distal apertures, and each arm of the cross brace assembly may have a proximal region including a proximal pin and a distal region including a distal pin. The proximal pins may be slidably disposed within the proximal slots and the distal pins may be rotatably disposed within the distal apertures. In some embodiments, the proximal slots and the distal apertures are disposed in side surfaces of the body members.

Each body member of the main body may include distal slots, and each arm of the cross brace assembly may have a barb. The barbs may be movable within the distal slots such that when the main body is disposed in the closed position, the barbs are disposed within the distal slots and when the main body is in the open position, the barbs extend laterally through the distal slots. In some embodiments, the distal slot is defined through inner and outer surfaces of each body member, and the barb is disposed at a distal-most end of each arm.

In accordance with another aspect of the present disclosure, a kit includes a dilator, a retraction device, and a scalpel. The dilator includes a main body having a pair of body members movably coupled to one another and movable in parallel relationship with respect to each other between a closed position and an open position. The retraction device is configured to engage the dilator and move the body members towards the open position.

In embodiments, the dilator further includes a cross brace assembly including two linkage assemblies. Each linkage assembly has a pair of arms arranged in an X configuration, and each arm includes at least one slidable connection point with the respective body member to which the arm is coupled to facilitate movement of the body members relative to each other.

In accordance with yet another aspect of the present disclosure, a method of accessing and retracting tissue includes pushing a main body of a dilator that is disposed in a closed position into an incision, and opening the main body of the dilator to an open position to retract the surrounding tissue. The main body of the dilator includes a pair of body members movably coupled to one another and movable in parallel relationship with respect to each other between the closed and open positions.

In embodiments, pushing the main body of the dilator may include advancing the main body into tissue until distal ends of the body members are disposed adjacent a vertebral body.

The method may include passing a threaded pin through a throughhole defined in the main body of the dilator and into the vertebral body, prior to opening the main body of the dilator.

In embodiments, opening the main body of the dilator includes connecting a retraction device to posts extending from the body members of the dilator, and operating the retraction device to move the body members to the open position.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure, wherein:

FIG. 1 is a perspective view of a scalpel of a surgical access system in accordance with an embodiment of the present disclosure;

FIG. 2A is a perspective view of a dilator of a surgical access system, in a closed position, in accordance with an embodiment of the present disclosure;

FIG. 2B is a perspective view of the dilator of FIG. 2A, with a threaded pin disposed within a main body of the dilator;

FIG. 3 is a perspective view of the dilator of FIGS. 2A and 2B in an open position, and in use with a retraction device of a surgical access system in accordance with an embodiment of the present disclosure;

FIG. 4A is a top view of the dilator of FIGS. 2A-3, in the open position of FIG. 3;

FIG. 4B is a side view of the dilator of FIG. 4A;

FIG. 4C is an end view of the dilator of FIGS. 4A and 4B;

FIG. 5 is a perspective view of a guidewire inserted into a disc of a spinal column;

FIG. 6A is a perspective view of a cannula and threaded pin for use with a spinal column in accordance with an embodiment of the present disclosure;

FIG. 6B is a perspective view of the threaded pin inserted through the cannula and into a vertebral body of the spinal column of FIG. 6A;

FIG. 6C is a perspective view of the threaded pin coupled to the vertebral body of the spinal column of FIG. 6B without the cannula;

FIG. 6D is a perspective view of the scalpel of FIG. 1, for use with the threaded pin of FIG. 6C;

FIG. 6E is a perspective view of the main body of the dilator of FIG. 2A, for use with the threaded pin of FIG. 6C;

FIG. 6F is a perspective view of the main body of the dilator inserted over the threaded pin of FIG. 6E;

FIG. 6G is a perspective view of the main body of the dilator of FIG. 6F in an open position, and in use with a retraction device; and

FIG. 6H is a top view of the dilator and the retraction device of FIG. 6G.

### DETAILED DESCRIPTION

Embodiments of the present disclosure are now described in detail with reference to the drawings in which like reference numerals designate identical or corresponding elements in each of the several views. The term “clinician” refers to a doctor (e.g., a surgeon), a nurse, or any other care provider, and may include support personnel. Throughout this description, the term “proximal” refers to a portion of a system, device, or component thereof that is closer to a clinician, and the term “distal” refers to the portion of the system, device, or component thereof that is farther from the clinician.

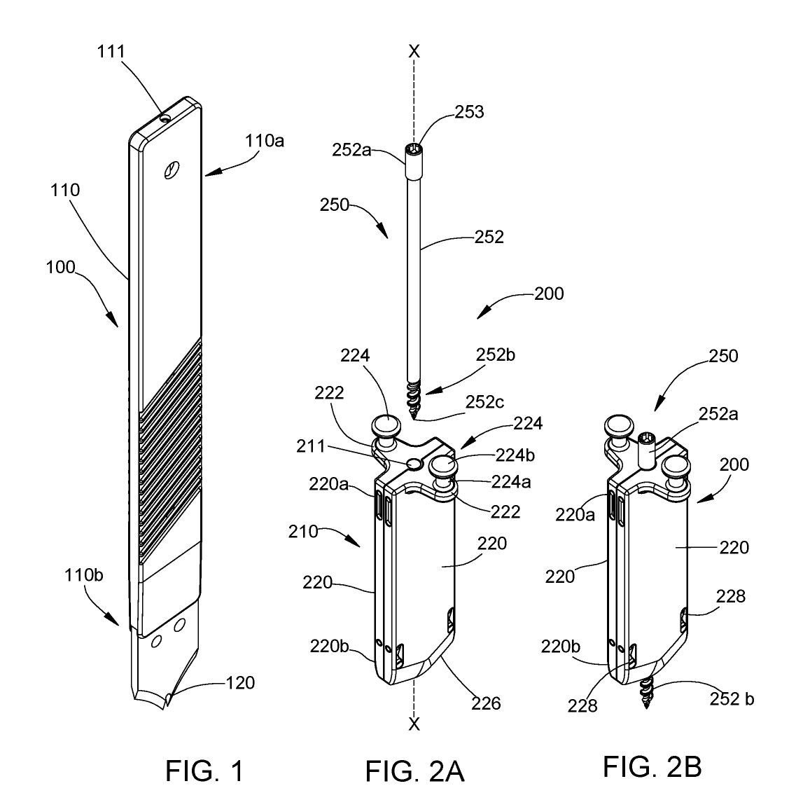

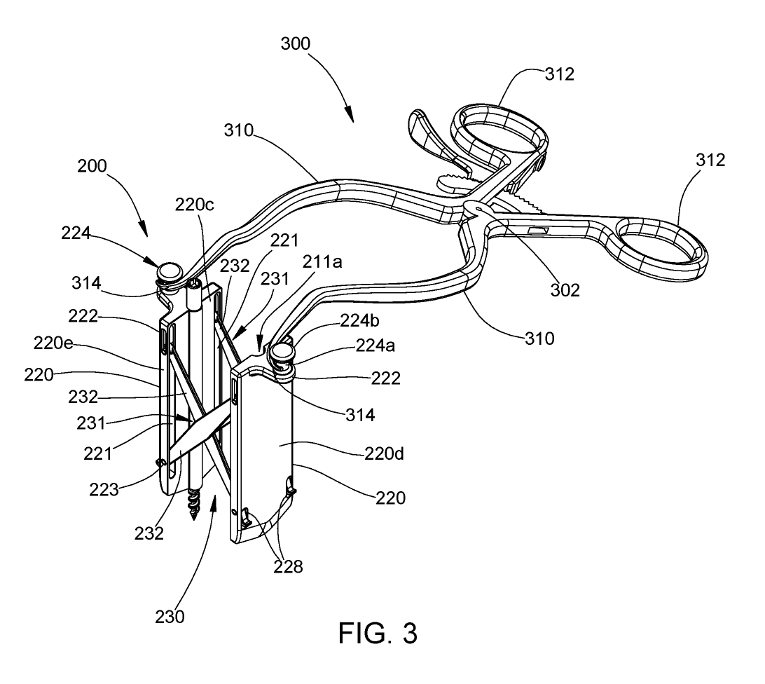

A surgical access system in accordance with the present disclosure includes a scalpel 100 (FIG. 1), a dilator 200 (see e.g., FIG. 2A), and a retraction device 300 (FIG. 3). The surgical access system is utilized to access, dilate, and retract tissue in a surgical site.

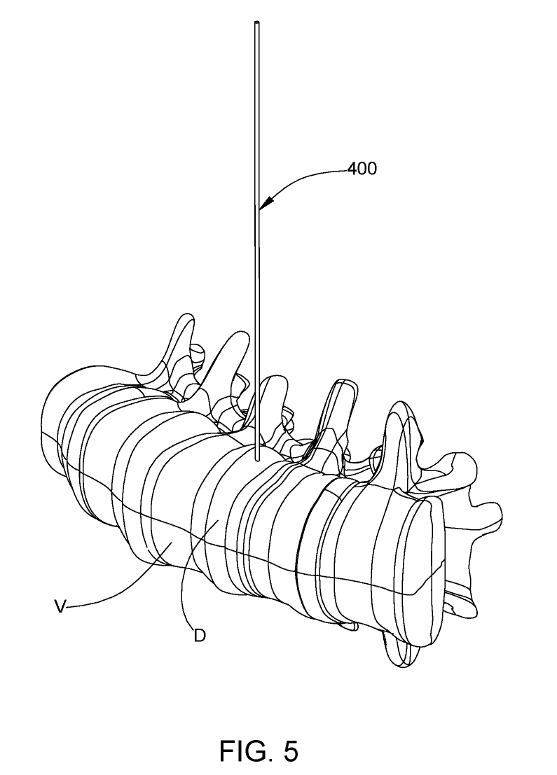

As shown in FIG. 1, the scalpel 100 includes a body 110 having a proximal region 110a defining a handle for grasping by a clinician, and a distal region 110b including a blade portion 120 for creating an incision through skin and/or fascia. In embodiments, the scalpel 100 is cannulated, and the body 110 includes a channel 111 defined therethrough for slidably receiving a guidewire 400 (FIG. 5).

Referring now to FIG. 2A, the dilator 200 includes a main body 210 and a threaded pin 250 that is removably received within a throughhole or passageway 211 defined in the main body 210 along a longitudinal axis “X.” The main body 210 includes body members 220 that are movable in parallel relationship with respect to each other between a closed configuration or position (see e.g., FIG. 2A) and an open configuration or position (see e.g., FIG. 3). Each body member 220 includes a proximal portion 220a and a distal portion 220b. The proximal portion 220a includes a flange 222 extending laterally therefrom, and a post 224 extending proximally from the flange 222. The post 224 includes a shaft 224a and an enlarged head 224b. The distal portion 220b of each body member 220 includes a tapered edge 226 tapering distally towards the longitudinal axis “X” (i.e., towards a centerline of throughhole 211) to facilitate insertion of the dilator 200 into tissue.

The threaded pin 250 includes an elongated body 252 having an enlarged proximal end 252a configured to facilitate driving the threaded pin 250 distally into bone, and a threaded distal end 252b terminating in a sharp distal tip 252c that is configured to penetrate or pierce bone. The proximal end 252a of the threaded pin 250 may include, for example, a hex shaped recess 253 configured to mate with an insertion instrument or driving tool (not shown), as is within the purview of those skilled in the art. The threaded pin 250 may be slid distally into the channel 211 of the main body 210 such that, as shown in FIG. 2B, the enlarged proximal end 252a abuts the proximal portions 220a of the body members 220 effectively acting as a stop, and the threaded distal end 252b extends out the distal portions 220b of the body members 220 for engagement with bone.

The dilator 200 is configured for use with a variety of retraction devices, such as hand-held retraction devices, like a Gelpi retractor, as shown, for example, in U.S. Pat. No. 7,846,093, the entire contents of which are hereby incorporated by reference herein, and retraction frames (e.g., table-mountable retraction frames), such as those shown in U.S. Pat. No. 8,449,463, the entire contents of which are hereby incorporated by reference herein.

As shown in FIG. 3, a Gelpi retractor 300 includes a pair of arcuate arms 310 that are pivotably connected at pivot point 302. A pair of finger rings 312 is located at a proximal end of the Gelpi retractor 300 that permit a clinician to selectively move the arms 310 towards and away from each other. A finger 314 is disposed at the distal end of each arm 310 and is configured to releasably engage a post 224 of the dilator 200. The fingers 314 curve around the shaft 224a of the respective post 224 and is retained between the enlarged head 224b of the post 224 and the flange 222 of the body member 220.

The Gelpi retractor 300 facilitates movement of the body members 220 of the dilator 200 laterally relative to the longitudinal axis “X” (FIG. 2A) of the dilator 200, thereby facilitating retraction of tissue that is disposed adjacent to outer surfaces 220d of the body members 220. The finger rings 312 of the Gelpi retractor 300 are laterally offset from the arms 310 such that pivotable movement of the arms 310 urge the body members 220 of the dilator 200 toward and away from each other in response to movement of the finger rings 312.

Moving the finger rings 312 towards each other pivots the arms 310 away from each other and urges the body members 220 away from each other, thereby separating tissue and enlarging a passageway therethrough. Consequently, movement of the finger rings 312 away from each other pivots the arms 310 towards each other, allowing the dilator 200 to return to the closed position. In embodiments, an outside force, such as pressure from the surrounding tissue or manual movement, aid in moving the body members 220 of the dilator 200 towards the closed position. In some embodiments, additional structure such as, for example, a biasing mechanism coupled to the body members 220 of the dilator 200 or fingers of a retraction device that engage outer surfaces or encircle the posts 224 of the body members 220, move the body members 220 of the dilator 200 to the closed position.

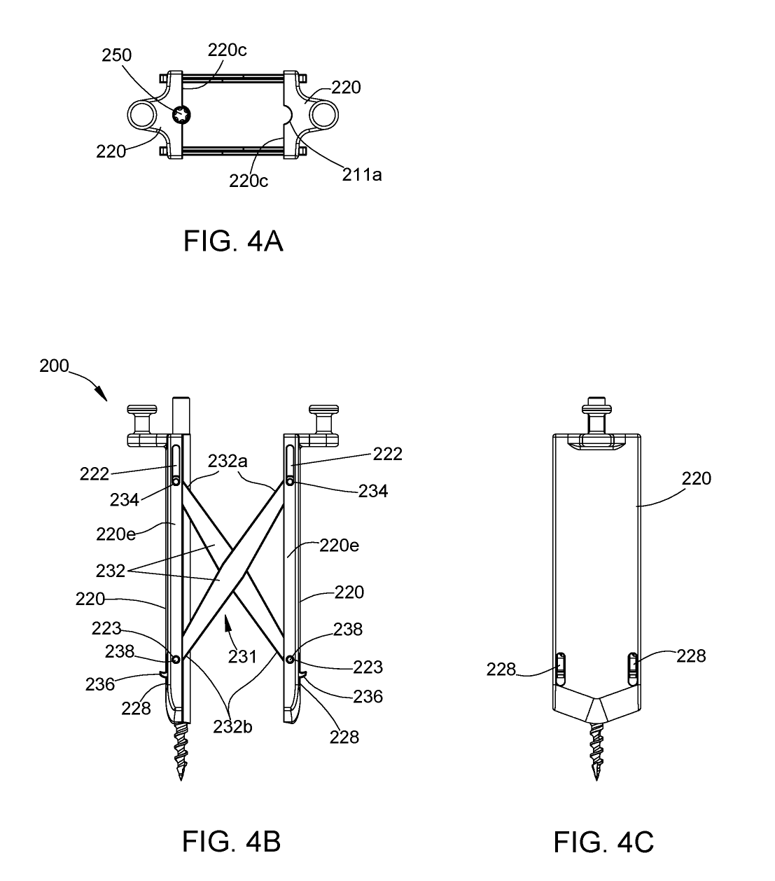

With continued reference to FIG. 3, each body member 220 includes an inner surface 220c, an outer surface 220d, and side surfaces 220e. A recess 211a is defined in each of the inner surfaces 220c of the body members 220 such that, when mated together in the closed position (see e.g., FIG. 2A), the recesses 211a define the throughhole 211 of the dilator 200. In embodiments, as shown in FIG. 4A, one of the recesses 211a is configured to retain the threaded pin 250 therein when the body members 220 are in the open configuration, and the other of the recesses 211a is configured to mate and support the threaded pin 250 when the body members 220 are in the closed position. In some embodiments, either recess 211a can receive and retain the threaded pin 250 therein.

The inner surface 220c of each body member 220 includes a pair of longitudinally extending grooves 221 defined therein. Each groove 221 is disposed adjacent a side surface 220e of the body member 220. As shown in FIG. 4B, in conjunction with FIG. 3, each side surface 220e of the body members 220 includes a proximal slot 222 and a distal aperture 223 defined therethrough that are in communication with the respective groove 221. As shown in FIG. 4C, in conjunction with FIG. 3, each body member 220 includes a pair of distal slots 228 defined through the inner and outer surfaces 220c, 220d thereof and disposed at distal regions of the grooves 221.

Referring again to FIGS. 3 and 4B, a cross brace assembly 230 interconnects the body members 220 of the dilator 200, controlling the movement (e.g., opening and closing) of the body members 220 with respect to each other, as well as maintaining the body members 220 in a parallel relationship with respect to each other. The parallel relationship is maintained throughout the movement of the body members 220, even under extreme distraction forces, providing uniform distraction/retraction of tissue relative to a centerline of the throughhole 211 of the dilator 200, and stability without cantilever bending and/or flexure of the body members 220. Additionally, the cross brace assembly 230 aids in holding tissue disposed adjacent the outer surfaces 220d of the body members 220 open when the dilator 200 is in an open position, providing additional support to the surrounding tissue and an increased field of view.

The cross brace assembly 230 includes two linkage assemblies 231. Each linkage assembly 231 includes arms 232. Each arm 232 has a proximal region 232a including a proximal pin 234, and a distal region 232b terminating in a barb 236 and including a distal pin 238. The proximal region 232a of each arm 232 is positioned within a groove 221 of a body member 220 with the proximal pin 234 inserted into, and slidable within, the respective proximal slot 222. The distal region 232b of each arm 232 is positioned within the groove 221 of the other of the body members 220 such that the arms 232 cross in an “X” configuration. The distal pin 238 of each arm 232 is inserted into the respective distal aperture 223 and rotatable about an axis of the distal pin 238, and the barb 236 of each arm 232 is inserted into, and slidable within, the respective distal slot 228. Accordingly, the arms 232 are rotatably fixed to the body members 220 about the distal pins 238, and movable about the proximal pins 234, which ride in the proximal slots 222 of the body members 220.

The barbs 236 of the arms 232 are movable in the distal slots 228 of the body members 220. The barbs 236 are configured to grab and engage tissue, providing an anchor for the dilator 200 when the dilator 200 is in the open position. As shown, for example, in FIG. 2B, when the dilator 200 is in the closed position, the barbs (not shown) are positioned within the distal slots 228 of the body members 220 to allow uninhibited passage of the dilator 200 through tissue. When the dilator 200 is in the open position, as shown, for example, in FIG. 4B, the barbs 236 extend laterally through the distal slots 228 to aid in retaining the dilator 200 in the open position in an incision.

In a method of use, the blade portion 120 of the scalpel 100 is utilized to create an incision or pathway in tissue for the dilator 200. The scalpel 100 is removed, and the main body 210 of the dilator 200, in the closed configuration, is pushed into the incision and through the created pathway until the distal portions 220b of the body members 220 of the dilator 200 are positioned adjacent to bone, such as a vertebral body.

Optionally, as shown in FIG. 5, a guidewire 400 may be used to facilitate the insertion of the scalpel 100 (FIG. 1) and/or dilator 200 (FIG. 2A) into tissue. The guidewire 400 may be positioned in a disc “D” by, for example, advancing a needle (not shown) into the disc “D,” advancing the guidewire 400 through the needle, and removing the needle over the guidewire 400, leaving the guidewire 400 in place in the disc “D.” The scalpel 100 (FIG. 1) is introduced over the guidewire 400 (through the channel 111 of the scalpel 100) to aid in guiding the scalpel 100 while creating the incision. The scalpel 100 is then removed over the guidewire 400, and the main body 210 (FIG. 2A) of the dilator 200 is introduced over the guidewire 400 (through the throughhole 211of the main body 210) to guide the dilator 200 to a vertebral body “V” adjacent the disc “D.” The guidewire 400 may then be removed. In embodiments, the guidewire 400 includes depth markings thereon at predetermined lengths to aid a clinician in determining the depth at which the guidewire 400 is inserted into the vertebral body “V.” The depth markings also indicate a distance between a distal tip of the guidewire 400 and an outer surface of body tissue.

After inserting the main body 210 of the dilator 200 adjacent the bone, the threaded pin 250 is inserted into the throughhole 211 of the dilator 200 and into the bone to facilitate retention of the dilator 200 therein. The pin 250 may be positioned, for example, adjacent a pedicle, an anterior face, or a lateral side of a vertebral body as shown. Other locations on a vertebral body are also contemplated. The posts 224 of the dilator 200 are then engaged, by a retraction device 300 (FIG. 3), to open the body members 220 of the dilator 200 and hold surrounding tissue open around the surgical site. The barbs 236 of the cross brace assembly 230 prevents expulsion of the dilator 200 from the surgical site by holding the dilator 200 within the soft tissue, along with the threaded pin 250 which holds the body members 220 adjacent the bone.

With the tissue separated, a clinician has increased visualization of the surgical site for performing a desired surgical procedure, such as a discectomy.

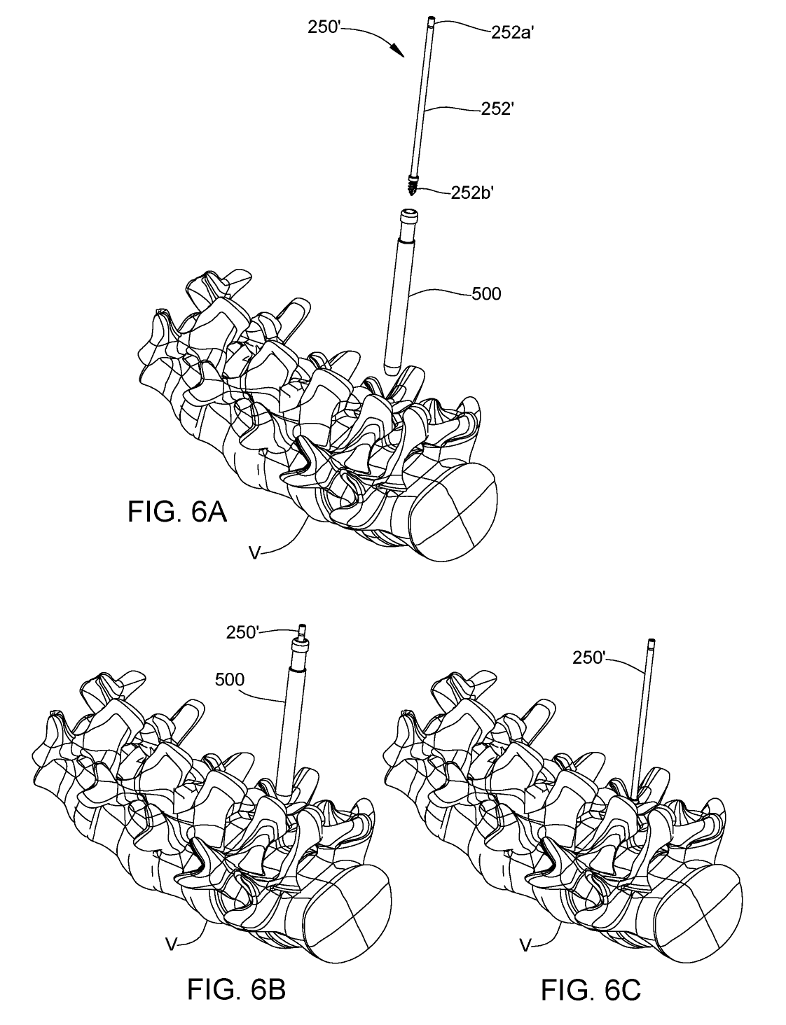

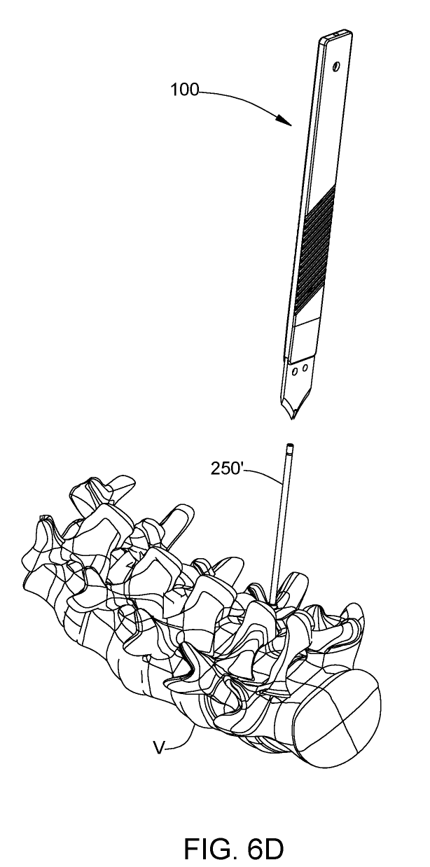

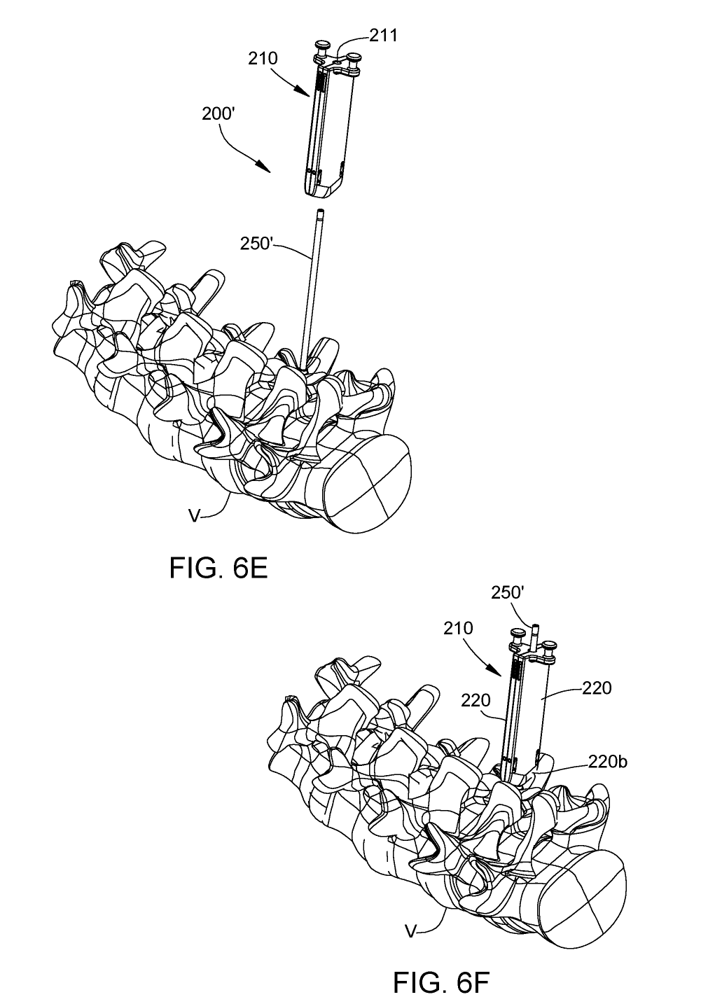

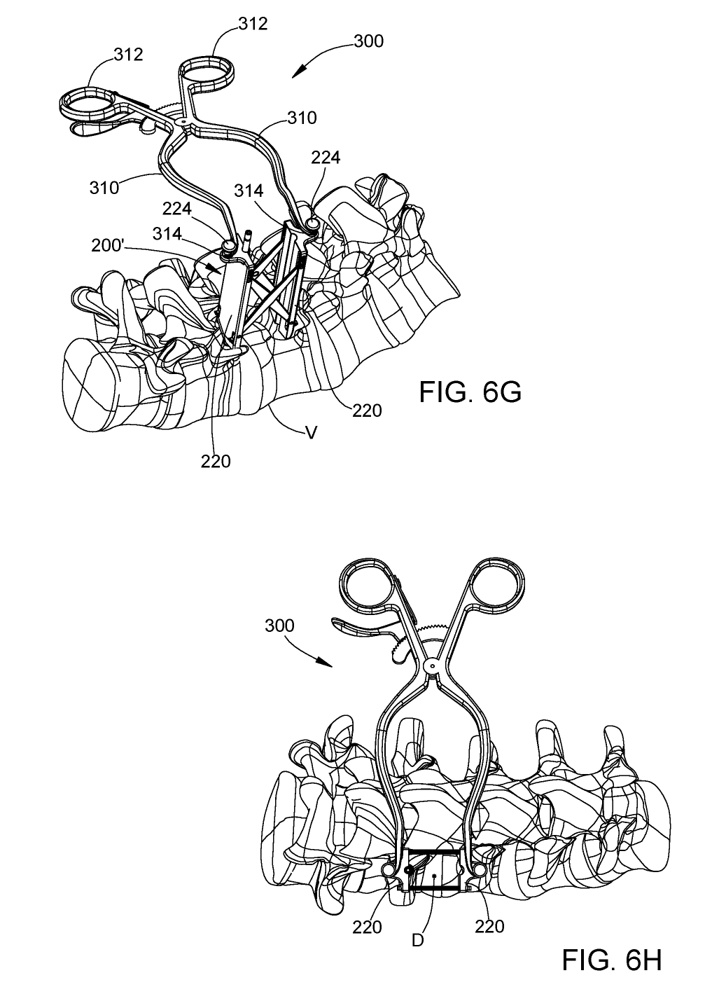

In another method of use, as shown in FIG. 6A, a cannula 500 is positioned through tissue (not shown) adjacent a vertebral body “V”, as is within the purview of those skilled in the art, and a threaded pin 250′ may be advanced through the cannula 500, as shown in FIG. 6B. The threaded pin 250′ is substantially similar to threaded pin 250, except that the proximal end 252a′ is not enlarged (i.e., a substantially uniform outer diameter) thereby allowing the entire elongated body 252′, including the proximal end 252a′ and the distal end 252b′, of the threaded pin 250′ to be passed through the cannula 500. The threaded distal end 252b′ is threaded into the vertebral body “V”, and the cannula 500 is removed over the threaded pin 250′, as shown in FIG. 6C. It is also contemplated that the threaded pin 250′ may be inserted into a vertebral body “V” prior to inserting the cannula 500. This allows the threaded pin 250′ to act as a guide for inserting one or more cannulae 500 over the threaded pin 250′. The threaded pin 250′ may be positioned, for example, adjacent a pedicle, an anterior face, or a lateral side of a vertebral body. Other locations on a vertebral body are also contemplated.

In embodiments, the threaded pin 250′ includes depth markings thereon at predetermined lengths to aid a clinician in determining the depth at which the threaded pin 250′ is inserted into the vertebral body “V.” The depth markings also indicate a distance between a distal tip of the threaded pin 250′ and an outer surface of body tissue. In some embodiments, additional cannulae (not shown) of increasing diameters may be subsequently inserted over one another to increase the size of the opening of the surgical site.

As shown in FIG. 6D, the scalpel 100 may be introduced over the threaded pin 250′ to create an incision or pathway in the tissue, as described above. After the scalpel 100 is removed, the throughhole 211 of the main body 210 of the dilator 200′ is aligned with the threaded pin 250′, as shown in FIG. 6E, and then inserted over the threaded pin 250′ until distal portions 220b of the body members 220 of the main body 210 are positioned adjacent the vertebral body “V,” as shown in FIG. 6F. The posts 224 of the dilator 200′ are then engaged by the fingers 314 of the retractor device 300, and the finger rings 312 of the retractor device 300 are manipulated by a clinician to open the arms 310 of the retractor device 300 and thus the body members 220 of the dilator 200′ to the open position, as shown in FIGS. 6G and 6H, separating surround tissue (not shown) for increased visualization and/or accessibility to the surgical site, such as disc “D.”

The spinal access system may be provided in a kit. The kit is an assembled package including a scalpel, a dilator, and a retraction device. In embodiments, the kit may include a plurality of scalpels, dilators, and/or retraction devices. For example, the scalpels may be cannulated or non-cannulated, have different blade configurations and/or different lengths, the dilators may have different sized body members and/or different open diameters, and/or the retractors may have different handle and/or finger configurations, to allow a clinician to pick and choose instruments for use in a desired surgical procedure.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. For example, the surgical access system of the present disclosure, and devices thereof, may be utilized to access and/or retract tissue at a surgical site in a variety of orthopedic surgery applications. Accordingly, the subject matter of the present disclosure is not limited by what has been particularly shown and described.

What is claimed is:

1. A dilator comprising:
a main body including a pair of body members movable in parallel relationship with respect to each other between a closed position and an open position; and
a cross brace assembly including two linkage assemblies, each linkage assembly having a pair of arms arranged in an X configuration, each arm including at least one slidable connection point with the respective body member to which the arm is coupled to facilitate movement of the body members relative to each other.

2. The dilator according to claim 1, wherein each body member includes a post releasably engageable with a retractor device configured to move the body members to the open position.

3. The dilator according to claim 2, wherein each body member includes a flange extending laterally from a proximal portion thereof, and the post extends proximally from the flange.

4. The dilator according to claim 3, wherein each post includes a shaft and an enlarged head.

5. The dilator according to claim 1, wherein a distal portion of each body member includes a tapered edge.

6. The dilator according to claim 1, further including a threaded pin having an elongated body removably disposed within a throughhole defined in the main body.

7. The dilator according to claim 6, wherein the elongated body of the threaded pin includes an enlarged proximal end and a threaded distal end, the threaded distal end extending distally beyond distal portions of the body members when the threaded pin is disposed in the throughhole.

8. The dilator according to claim 6, wherein each body member includes an inner surface having a recess defined therein that together define the throughhole of the main body when in the closed position, one of the recesses configured to retain the threaded pin therein when the body members are in the open position.

9. The dilator according to claim 1, wherein each body member of the main body includes proximal slots and distal apertures, and each arm of the cross brace assembly has a proximal region including a proximal pin and a distal region including a distal pin, the proximal pins slidably disposed within the proximal slots and the distal pins rotatably disposed within the distal apertures.

10. The dilator according to claim 9, wherein the proximal slots and the distal apertures are disposed in side surfaces of the body members.

11. The dilator according to claim 9, wherein each body member of the main body includes distal slots, and each arm of the cross brace assembly has a barb, the barbs movable within the distal slots such that when the main body is disposed in the closed position, the barbs are disposed within the distal slots and when the main body is in the open position, the barbs extend laterally through the distal slots.

12. The dilator according to claim 11, wherein the distal slot is defined through inner and outer surfaces of each body member, and the barb is disposed at a distal-most end of each arm.

13. A kit comprising:
a dilator including:
a main body including a pair of body members movably coupled to one another and movable with respect to each other between a closed position and an open position;
a retraction device configured to engage the dilator and move the body members to the open position; and
a scalpel.

14. The kit according to claim 13, wherein the dilator further includes a cross brace assembly including two linkage assemblies, each linkage assembly having a pair of arms arranged in an X configuration, each arm including at least one slidable connection point with the respective body member to which the arm is coupled to facilitate movement of the body members relative to each other.

15. The kit according to claim 13, wherein the body members are movable in a parallel relationship with respect to each other.

16. A method of accessing and retracting tissue, comprising:
pushing a main body of a dilator disposed in a closed position into an incision, the main body including a pair of body members movably coupled to one another and movable with respect to each other between the closed position and an open position; and
opening the main body of the dilator to the open position to retract surrounding tissue.

17. The method according to claim 16, wherein pushing the main body of the dilator includes advancing the main body into tissue until distal ends of the body members are disposed adjacent a vertebral body.

18. The method according to claim 17, further comprising:
passing a threaded pin through a throughhole defined in the main body of the dilator and into the vertebral body, prior to opening the main body of the dilator.

19. The method according to claim 16, wherein opening the main body of the dilator includes connecting a retraction device to posts extending from the body members of the dilator, and operating the retraction device to move the body members to the open position.

20. The method according to claim 16, wherein pushing the main body of the dilator includes the pair of body members movable with respect to each other in a parallel relationship.
